(19) United States
(12) Patent Application Publication
(10) Pub. No.: US 2012/0149962 A1
(43) Pub. Date: Jun. 14, 2012

(54) IN SITU REMOVAL OF IRON COMPLEXES DURING CRACKING

(76) Inventors: Vasily Simanzhenkov, CALGARY (CA); Oleksiy Golovchenko, CALGARY (CA); Andrzej Krzywicki, CALGARY (CA)

(21) Appl. No.: 13/293,259

(22) Filed: Nov. 10, 2011

(30) Foreign Application Priority Data
Dec. 8, 2010 (CA) 2724389

(51) Int. Cl. C07C 4/02

(52) U.S. Cl. 585/648

(57) ABSTRACT

The presence of complexes predominantly of iron and one or more of chromium, nickel and oxygen and mixtures thereof on the surface of a stainless steel exposed to a feed stream containing hydrocarbons at elevated temperatures tends to give rise to decomposition products of the hydrocarbon. The amount of iron complexes may be reduced in situ without stopping the process by adding to the feed stream 0.001 to 1 vol % a silane and optionally from 0 to 500 ppm based on the weight of the feed stream of sulphur or a sulphur containing compound.

### FIELD OF THE INVENTION

The present invention relates to the removal of complexes of iron from the internal surface of equipment used to treat aliphatic hydrocarbons at high temperatures such as steam crackers. More particularly the present invention relates to a method to reduce deposits of mixtures and complexes comprising iron and one or more of chromium, nickel and oxygen on the internal surface of high chrome and high nickel furnace tubes.

### BACKGROUND OF THE INVENTION

There is an increasing amount of art discussing the formation of protective surfaces of metallic complexes or mixtures typically comprising two or more elements selected from the group consisting of Cr, Mn, Ni, Al and O. These complexes or mixtures do not comprise Fe. These complexes or mixtures tend to provide a protective coating either in terms of reducing corrosion or in terms of reducing fouling such as coking. This type of technology may be used for example on surfaces of high temperature reactors for treating hydrocarbons, preferably aliphatic hydrocarbons be it naphtha or ethane and propane feeds. Particularly these feeds may be cracked to olefins.

U.S. Pat. No. 7,156,979 issued Jan. 2, 2007 in the name of Benum et al, assigned to NOVA Chemicals (International) S.A. teaches increased run length when the internal surface of a high nickel high chromium furnace tube has a coating of the formula MnCr₂O₄.

Great Britain patent application 2,159,542 published Dec. 4, 1985 in the name of Zeilinger assigned to Man Machinenfabrik Augsburg Nurnberg AG discloses somewhat similar types of coatings.

U.S. Pat. No. 4,976,932 issued Dec. 11, 1990 to Maeda et al assigned to JGC Corporation is comparable.

U.S. Pat. No. 7,396,597 issued Jul. 8, 2008 to Nishiyama et al, assigned to Sumitomo Metal Industries, Ltd. is comparable.

The above art suggests that the presence of iron, iron oxides, iron complexes and mixtures with other metals at the surface of an alloy exposed to hydrocarbons at an elevated temperature is not desirable. Industrial processes being what they are iron may make its way to the type of surface described above. This has a number of disadvantages. The iron or complex or mixture may be a site for carburization of the hydrocarbon. The iron could provide a site for scaling or spalling of the protective surface. In any event the iron compound complex or mixture needs to be removed from the surface or at least reduced.

Preferably this should be done without having to stop the process.

There are a number of patents which teach the pretreatment of furnace tubes or coils with various compositions containing silicon and additional elements. These include the following patents or families of patents.

U.S. Pat. No. 7,604,730 issued Oct. 20, 2009 to Humblot et al, assigned to Arkema France.

Canadian patent 2,152,336 published Feb. 26, 1996 to Degraffenriedd, et al assigned to Phillips Petroleum Company, now abandoned.

U.S. Pat. No. 6,464,858 issued Oct. 15, 2002 to Brown et al. assigned to Phillips Petroleum Company. In addition to teaching a pretreatment of coils the patent is directed to heavier feedstocks.

U.S. Pat. No. 5,922,192 issued Jul. 13, 1999 to Zimmermann et al. assigned to Mannesmann Aktiengesellschaft.

U.S. Pat. No. 4,692,234 issued Sep. 8, 1987 to Porter et al. assigned to Phillips Petroleum Company. The patent teaches pre and continuous treatment of furnace tubes with silicon and one or more of tin and antimony. The present invention does not contemplate the use of either tin or antimony.

U.S. Pat. No. 5,658,452 issued Aug. 19, 1997 to Heyse et al assigned to Chevron Chemical Company. This patent teaches painting, cladding or plating mixtures of silicon and other metallic coke inhibitors to furnace tubes prior to cracking to reduce the amount of steam needed in the process and to reduce coking.

U.S. Pat. No. 5,413,813 issued May 9, 1995 to Cruse et al assigned to Enichem S.p.A. discloses a chemical vapor deposition (CVD) process in which a silica containing compound, typically a silazine, is decomposed and the resulting vapor is deposited as a ceramic in the furnace tubes. This results in an inert ceramic lining on the inner surface of the tube. This is done prior to cracking. The reference does not teach the reduction of iron impurities at the internal surface of the cracking tube.

U.S. Pat. No. 5,208,069 issued May 4, 1993 to Clark et al., assigned to Enichem S.p.A. and Istituto Guido Donegani S.p.A. teaches forming a ceramic on the inner surface of a furnace tube by the vapor deposition of a ceramic precursor. The ceramic precursor is carried through the furnace tube in an inert gas. Suitable inert gas may be selected from the group consisting of nitrogen, argon, helium, methane, ethylene, ethane, hydrogen and mixtures thereof. Minor amounts of oxygen or oxygen-containing gases, such as carbon dioxide and monoxide, do not impair the properties of the obtained coating. The patent teaches against the presence of steam as a carrier for the silicon compound.

Chinese patent 100497529 published Mar. 14, 2007 in the name of Xu Hong Zhou, assigned to the University of East China Science and Technology teaches the addition of mixtures of sulfur, magnesium and silicon after coke is removed from furnace tubes before bringing the tubes back into service. This is not an ongoing process but rather is carried out after decoking. Additionally, the present invention does not contemplate the use of magnesium.

U.S. Pat. No. 5,567,305 issued Oct. 22, 1996 to Jo teaches the continuous addition to the hydrocarbon feed stock in the coil at the end of the convection stage of the pyrolysis furnace a mixture of Group IA metal salt, a Group IIA metal salt, an aluminum compound and a silicon compound. This is to reduce coking and corrosion in the furnace tubes and the transfer line exchangers. The present invention has eliminated the Group IA metal salt, a Group IIA metal salt, and aluminum compound.

The present invention seeks to provide a simple means of ameliorating deposits of iron and one or more metals or oxides on a protective surface on a steel substrate used to treat a hydrocarbon at elevated temperatures.

### SUMMARY OF THE INVENTION

The present invention provides a method to reduce deposits of mixtures, complexes, or both comprising predominantly iron and one or more of chromium, nickel and oxygen and mixtures thereof on the internal surface of a furnace tube comprising 20 to 65 wt % of Ni and 10 to 50 wt % of Cr during the cracking of a C₂₋₄parafin feed comprising adding from 0.001 to 1 vol % based on the total volume of the feed stream of a silane of the formula (Si)ₙR₂ₙ₊₂ where R is selected from the group consisting of a hydrogen atom and alkyl or aromatic radicals and optionally from 0 to 500 ppm based on the weight of the feed stream of sulphur or a sulphur containing compound to the feed stream.

In a further embodiment in the silane all of the R substituents are the same.

In a further embodiment the feed stream comprise steam and a C₂₋₄ paraffin in a weight ratio of steam to ethane from 0.25:1 to 40:1.

In a further embodiment the cracking takes place at a temperature from 650° C. to 1100° C.

In a further embodiment the iron mixtures, complexes, or both are selected from the group consisting of FeCr₂O₄, Ni_{2.9}Cr_{0.7}Fe_{0.36}, Fe₂(CrO₄)₃, Fe₂(Cr₂O₇)₃.

In a further embodiment in the silane R is selected from the group consisting of hydrogen, methyl and phenyl.

In a further embodiment the cracking takes place at a temperature from 800° C. to 1050° C.

In a further embodiment the C₂₋₄ paraffin is selected from the group consisting of ethane, propane and mixtures thereof.

In a further embodiment in the silane R is hydrogen.

In a further embodiment the furnace tube substrate comprises from about 55 to 65 weight % of Ni; from about 20 to 10 weight % of Cr; from about 20 to 10 weight % of Co; and from about 5 to 9 weight % of Fe and the balance one or more of the trace elements.

In a further embodiment the trace elements comprise from 0.2 up to 3 weight % of Mn, from 0.3 to 2 weight % of Si; less than 5 weight % of titanium, niobium and all other trace metals; and carbon in an amount of less than 0.75 weight % the sum of the components adding up to 100 weight %.

In the above embodiment of the invention the inner surface of the furnace tube may comprise a surface layer from 1 to 50 microns thick comprising from 90 to 10 weight % of a spinel of the formula MnₓCr₃₋ₓO₄ wherein x is from 0.5 to 2, from 10 to 90 weight % of oxides of Mn, Si selected from the group consisting of MnO, MnSiO₃, Mn₂SiO₄ and mixtures thereof.

In a further embodiment the furnace tube substrate comprises comprise from 40 to 65 weight % of Co; from 15 to 20 weight % of Cr; from 20 to 13 weight % of Ni; less than 4 weight % of Fe and the balance of one or more trace elements and up to 20 weight % of W the sum of the components adding up to 100 weight %.

In the embodiment noted above the trace elements comprise from 0.2 up to 3 weight % of Mn, from 0.3 to 2 weight % of Si; less than 5 weight % of titanium, niobium and all other trace metals; and carbon in an amount of less than 0.75 weight %

In the above embodiment of the invention the inner surface of the furnace tube may comprise a surface layer from 1 to 50 microns thick comprising from 90 to 10 weight % of a spinel of the formula MnₓCr₃₋ₓO₄ wherein x is from 0.5 to 2, from 10 to 90 weight % of oxides of Mn, Si selected from the group consisting of MnO, MnSiO₃, Mn₂SiO₄ and mixtures thereof.

In a further embodiment the furnace tube substrate comprises from 20 to 38 weight % of chromium and from 25 to 48 weight % of Ni.

In a further embodiment the furnace tube substrate further comprises from 0.2 up to 3 weight % of Mn, from 0.3 to 2 weight % of Si; less than 5 weight % of titanium, niobium and all other trace metals; and carbon in an amount of less than 0.75 weight and the balance substantially iron.

In a further embodiment not less than 50% of the inner surface of the furnace tube a surface layer from 1 to 25 microns thick comprising a spinel of the formula MnCr₂O₄.

In a further embodiment of the invention the inner surface of the furnace tube may comprise a surface layer from 1 to 50 microns thick comprising from 90 to 10 weight % of a spinel of the formula MnₓCr₃₋ₓO₄ wherein x is from 0.5 to 2, from 10 to 90 weight % of oxides of Mn, Si selected from the group consisting of MnO, MnSiO₃, Mn₂SiO₄ and mixtures thereof.

The present invention includes combinations in whole or in part of the foregoing embodiments together with disclosures in the following specification.

### DETAILED DESCRIPTION

Typically, in accordance with the present invention the substrate steel may be any material to which a composite protective coating as referenced above such as Cr₂O₃ or Mnr₂O₄ and the like will bond. The substrate may be a carbon steel or a stainless steel which may be selected from the group consisting of wrought stainless, austentic stainless steel and HP, HT, HU, HW and HX stainless steel, heat resistant steel, and nickel based alloys. The substrate may be a high strength low alloy steel (HSLA); high strength structural steel or ultra high strength steel. The classification and composition of such steels are known to those skilled in the art.

In one embodiment the stainless steel, preferably heat resistant stainless steel typically comprises from 13 to 50, preferably 20 to 50, most preferably from 20 to 38 weight % of chromium. The stainless steel may further comprise from 20 to 50, preferably from 25 to 50 most preferably from 25 to 48, desirably from about 30 to 45 weight % of Ni. The balance of the stainless steel may be substantially iron.

The present invention may also be used with nickel and/or cobalt based extreme austentic high temperature alloys (HTAs). Typically the alloys comprise a major amount of nickel or cobalt. Typically the high temperature nickel based alloys comprise from about 50 to 70, preferably from about 55 to 65 weight % of Ni; from about 20 to 10 weight % of Cr; from about 20 to 10 weight % of Co; and from about 5 to 9 weight % of Fe and the balance one or more of the trace elements noted below to bring the composition up to 100 weight %. Typically the high temperature cobalt based alloys comprise from 40 to 65 weight % of Co; from 15 to 20 weight % of Cr; from 20 to 13 weight % of Ni; less than 4 weight % of Fe and the balance one or more trace elements as set out below and up to 20 weight % of W. The sum of the components adding up to 100 weight %.

In some embodiments of the invention the substrate may further comprise at least 0.2 weight %, up to 3 weight % typically 1.0 weight %, up to 2.5 weight % preferably not more than 2 weight % of manganese; from 0.3 to 2, preferably 0.8 to 1.6 typically less than 1.9 weight % of Si; less than 3, typically less than 2 weight % of titanium, niobium (typically less than 2.0, preferably less than 1.5 weight % of niobium) and all other trace metals; and carbon in an amount of less than 2.0 weight %. The trace elements are present in amounts so that the composition of the steel totals 100 wt %.

The outermost surface of the stainless steel has a thickness from 0.1 to up to 50, preferably from 0.1 to 25, most preferably from 0.1 to 10 microns and is a spinel of the formula MnₓCr₃₋ₓO₄ wherein x is from 0.5 to 2. Generally, this outermost spinel surface covers not less than 55%, preferably not less than 60%, most preferably not less than 80%, desirably not less than 95% of the stainless steel.

The spinel has the formula MnₓCr₃₋ₓO₄ wherein x is from 0.5 to 2. x may be from 0.8 to 1.2. Most preferably x is 1 and the spinel has the formula MnCr₂O₄.

In other embodiments of the invention the outermost (internal surface of the furnace tube) surface of the stainless steel may comprise from 90 to 10 weight %, preferably from 60 to 40 weight %, most preferably from 45 to 55 weight % the spinel (e.g. MnₓCr₃₋ₓO₄ wherein x is from 0.5 to 2 and from 10 to 90 weight %, preferably from 40 to 60 weight %, most preferably from 55 to 45 weight % of oxides of Mn, Si selected from the group consisting of MnO, MnSiO₃, Mn₂SiO₄ and mixtures thereof).

If the oxide in the surface has a nominal stoichiometry of MnO the Mn may be present in the surface in an amount from 1 to 50 atomic %. Where the oxide in the surface is MnSiO₃, the Si may be present in the surface in an amount from 1 to 50 atomic %. If the oxide in the surface is Mn₂SiO₄, the Si may be present in the surface in an amount from 1 to 50 atomic %.

The surface compositions should comprise less than 5, preferably less than 2, most preferably less than 0.5 weight % of Cr₂O₃. Most preferably Cr₂O₃ is absent in the surface or the compositions used to prepare the surface.

There are a number of manners in which the internal surface of the furnace tube (i.e. the radiant heated section) may become contaminated with mixtures, complexes, or both comprising predominantly iron and one or more of, chromium, nickel and oxygen and mixtures thereof. One contaminate may be iron oxide (either Fe₂O₃ or Fe₃O₄ or a mixture thereof). This is most likely to arise from up stream iron contamination of the feed stock or by spalling/exfoliation of the coating on the internal surface of the furnace tube. Iron and chrome complexes may be formed as chrome oxides may be present on the internal surface of the furnace tube (e.g. FeCr₂O₄, Fe₂(CrO₄)₃, Fe₂(Cr₂O₇)₃) or a mixture of the iron and chrome complexes (oxides) could be formed. Similarly nickel may be exposed on the surface of the furnace tube due to spalling/exfoliation of the coating. The presence of nickel and iron together could result in a complex or a mixed oxide (such as Ni₂FeO₄) or mixtures thereof. The presence of nickel, chromium and iron may result in a metallic complex (which need not be stoichometric) for example the complexes could be of the formula NiₐCr_{b}Fe_{c} wherein a is a number between 2 and 3, preferably between 2.5 and 3, b is a number between 0.5 and 1, preferably between 0.6 and 0.75 and c is a number between 0.3 and 0.5, preferably between 0.3 and 0.4. Preferably the sum of a+b+c+ is from 3.14 to 4.25, preferably from 3.9 to 4.1 (e.g. Ni_{2.9}Cr_{0.7}Fe_{0.36})

Steam cracking may be carried out at temperatures from 650° C. to 1100° C., preferably from 800° C. to 1050° C. The paraffin feed may be selected from the group consisting of C₂₋₄ paraffins, preferably ethane and propane. Steam is present in the feed stream to the cracker in an amount to provide a weight ratio of steam to paraffin from 0.25:1 to 40:1, typically from about 5:1 to 30:1, preferably from 10:1 to 25:1.

The organo-silicone may be added to the feed stream in an amount from 0.001 to 1 vol. %, preferably 0.01 to 0.9 vol %, desirably from 0.25 to 0.75 vol. % based on the total volume of the feed stream.

The organo-silicone has the formula (Si)ₙR₂ₙ₊₂ where R is selected from the group consisting of a hydrogen atom and alkyl or aromatic radicals to the feed stream. Preferably R is selected from the group consisting of hydrogen, C₁₋₄ alkyl radicals and C₆₋₁₀ aromatic radicals, most preferably R is selected from the group consisting of hydrogen, methyl and phenyl radicals.

Optionally sulphur or a compound, preferably organic, containing or generating sulphur may be added to the feed stream to the cracker. Generally the sulphur or sulphur generating compound is added to the ethane. The sulphur or sulphur generating compound may be added to the feed stream in amounts from 0 (e.g. optionally) up to 500 ppm based n the total weight of the feed. If present, the sulphur or sulphur containing or generating compound may be used in amounts to provide from from 20 to 400, preferably from about 50 to 300 ppm by weight based on the total weight of the feed stream. The sulphur containing compound should not contain silicone. The sulphur containing compounds may have the formula R¹S_{X}R² where in R¹ and R² are independently selected from the group consisting of a hydrogen atom; C₁₋₄ alkyl radicals; and C₆₋₁₀ aromatic radicals provided that R¹ and R² may be taken together to form a cyclic structure (e.g. thiophene or benzothiophene) and x is an integer greater than or equal to 1. Some non limiting examples of sulphur compounds include alkyl mercaptans, dialkyl sulphides, dialkyl disulphides, dialkyl polysulphides and thiophene and benzothiophene. Preferably the sulphur compound is selected from the group consisting of hydrogen sulphide dimethyl sulphide, diethyl sulphide, preferably dimethyl disulphide.

The present invention is illustrated by the following non limiting examples.

### Set Up:

In the examples a technical scale quartz furnace was used as described in U.S. Pat. No. 6,772,771.

The Quartz Reactor Unit (QRU) is composed of three zones of equal dimensions. Typically, hydrocarbon feeds and where required air, and nitrogen and silane are introduced into the reactor inlet through a flow control system. A metering pump delivers the required water for steam generation into the tubular quartz reactor at the end of zone 1 of the furnace. The organo silicon (SiH₄) is premixed with the ethane prior to injection into the furnace. The vaporized hydrocarbon stream enters the reactor heated to 650° C., where steam cracking of the hydrocarbons takes place to make pyrolysis products. The space in the tubular reactor located between zone 2 and 3 of the furnace is known to have the most uniform temperature distribution profile. Every quartz boat containing metal coupons is calibrated to be in this specific location. Coupons are weighed before and after an experiment to determine the weight changes and the coupon surfaces can be examined by various instruments for morphology and surface composition. After the transfer line exchanger (the open part of the quartz tube past the furnace outlet), the process stream enters a product knockout vessel where gas and liquid effluents can be collected for further analyses or venting.

For decoke simulations air enters at a controlled flow rate of 2 standard liters per minute (slpm), replacing hydrocarbon feeds, through the feed delivery system. Water is also admitted, through the metering pump, into the preheater where steam is generated. The tubular furnace operates typically at 950° C.

### EXAMPLES

### Example 1

A sample of a commercial furnace tube comprising from about 20 to 38 weight % of chromium, from about 30 to 45 weight % of Ni the balance trace components and iron. The furnace tube had been treated to produce an internal protective surface comprising MnCr₂O₄ (spinel) which is largely resistant to coke formation. During commercial operation the protective coating had been damaged and there was an iron deposition on the surface of the steel.

Coupons of the sample having iron depositions on the spinel were placed in quartz boats which were placed in the furnace. The samples were subjected to two cycles of cracking in a stream comprising 1 vol % of organo silicon in a steam ethane, mixture having a steam:ethane weight ratio of 0.33:1 for 4 hours and a 1 hour decoke at 950° C. in an steam to air mixture have a steam:air weight ratio of 3:1.

The sample was analyzed before and after the treatment. The results are shown in table 1.

TABLE 1

| Element | Before Wt. % | After Wt. % |
|---|---|---|
| Cr | 47.3 | 66.5 |
| Mn | 11.9 | 13.2 |
| Ni | 13.2 | 3.4 |
| Si | 1.1 | 12.4 |
| Nb | 1.0 | 0.0 |
| Fe | 25.6 | 4.5 |
| Relative Fe loss | | 82 |

The experiment shows that the treatment reduced the iron which was deposited on the internal surface of the furnace tube by more than 80% without an adverse effect on the Cr, and Mn which forms the protective spinel coating on the inner surface of the furnace tube. The silicon content on the surface increased to about 12 wt %.

A surface x-ray analysis of the sample before and after treatment was carried out. The results are shown in Table 2.

TABLE 2

| | Quartz | FeCr₂O₄ | Ni_{2.9}Cr_{0.7}Fe_{0.36} | MnCr₂O₄ | Cr₂O₃ |
|---|---|---|---|---|---|
| Before | | 59.0 | 18.6 | 11.8 | 10.6 |
| Wt % | | | | | |
| After | 1.7 | 41.6 | 25.0 | 11.7 | 20.0 |
| Wt % | | | | | |

The analysis shows a significant phase shift has occurred as a result of the treatment. A significant proportion of the iron chromate has been decomposed with an increase in the chrome oxide layer.

Without being bound by theory it is believed that the organo-silicone compound decomposes under steam cracking conditions forming silanol groups (Si—OH) reacts preferentially with the iron to form iron silenols which appear to be less strongly bound to the surface of the stainless steel than the other surface components. The iron silanols appear to be easily removed in the gas stream over the surface of the coil.

What is claimed is:

1. A method to reduce deposits of mixtures, complexes, or both comprising predominantly iron and one or more of chromium, nickel and oxygen and mixtures thereof on the internal surface of a furnace tube comprising 20 to 65 wt % of Ni and 10 to 50 wt % of Cr during the cracking of a C₂₋₄parafin feed comprising adding from 0.001 to 1 vol % based on the total volume of the feed stream of a silane of the formula (Si)ₙR₂ₙ₊₂ where R is selected from the group consisting of a hydrogen atom and alkyl or aromatic radicals and optionally from 0 to 500 ppm based on the weight of the feed stream of sulphur or a sulphur containing compound to the feed stream.

2. The method according to claim 1, wherein in the silane all of the R substituents are the same.

3. The method according to claim 2, wherein the feed stream comprise steam and a C₂₋₄ paraffin in a weight ratio of steam to ethane from 0.25:1 to 40:1.

4. The method according to claim 3, wherein the cracking takes place at a temperature from 650° C. to 1100° C.

5. The method according to claim 4 wherein the iron mixtures, complexes, or both are selected from the group consisting of FeCr₂O₄, Fe₂(CrO₄)₃, Fe₂(Cr₂O₇)₃ Ni₂FeO₄, and NiₐCr_{b}Fe_{c} wherein a is a number between 2 and 3, b is a number between 0.5 and 1, and c is a number between 0.3 and 0.4.

6. The method according to claim 5, wherein in the silane R is selected from the group consisting of hydrogen, methyl and phenyl.

7. The method according to claim 6, wherein the cracking takes place at a temperature from 800° C. to 1050° C.

8. The method according to claim 7, wherein the C₂₋₄ paraffin is selected from the group consisting of ethane, propane and mixtures there of.

9. The method according to claim 8, wherein in the silane R is hydrogen.

10. The method according to claim 8, wherein the furnace tube substrate comprises from about 55 to 65 weight % of Ni; from about 20 to 10 weight % of Cr; from about 20 to 10 weight % of Co; and from about 5 to 9 weight % of Fe and the balance one or more of the trace elements.

11. The method according to claim 10 wherein the trace elements comprise from 0.2 up to 3 weight % of Mn; from 0.3 to 2 weight % of Si; less than 5 weight % of titanium, niobium and all other trace metals; and carbon in an amount of less than 0.75 weight % the sum of the components adding up to 100 weight %.

12. The method according to claim 11, wherein the inner surface of the furnace tube comprises a surface layer from 1 to 50 microns thick comprising from 90 to 10 weight of a spinel of the formula MnₓCr₃₋ₓO₄ wherein x is from 0.5 to 2, from 10 to 90 weight of oxides of Mn, Si selected from the group consisting of MnO, MnSiO₃, Mn₂SiO₄ and mixtures thereof.

13. The method according to claim 8, wherein the furnace tube substrate comprises from 40 to 65 weight % of Co; from 15 to 20 weight % of Cr; from 20 to 13 weight % of Ni; less than 4 weight % of Fe and the balance of one or more trace elements and up to 20 weight % of W the sum of the components adding up to 100 weight %.

14. The method according to claim 13, wherein the trace elements comprise from 0.2 up to 3 weight % of Mn; from 0.3 to 2 weight % of Si; less than 5 weight % of titanium, niobium and all other trace metals; and carbon in an amount of less than 0.75 weight %

15. The method according to claim 14, wherein the inner surface of the furnace tube comprises a surface layer from 1 to 50 microns thick comprising from 90 to 10 weight % of a spinel of the formula MnₓCr₃₋ₓO₄ wherein x is from 0.5 to 2, from 10 to 90 weight % of oxides of Mn, Si selected from the group consisting of MnO, MnSiO₃, Mn₂SiO₄ and mixtures thereof.

16. The method according to claim 8, wherein the furnace tube substrate comprises from 20 to 38 weight % of chromium from 25 to 48, weight % of Ni.

17. The method according to claim 16 wherein the furnace tube substrate further comprises from 0.2 up to 3 weight % of Mn, from 0.3 to 2 weight % of Si; less than 5 weight % of titanium, niobium and all other trace metals; and carbon in an amount of less than 0.75 weight % and the balance substantially iron.

18. The method according to claim 17, wherein not less than 50% of the inner surface of the furnace tube is a surface layer from 1 to 50 microns thick comprising a spinel of the formula MnCr₂O₄.

19. The method according to claim 17, wherein the inner surface of the furnace tube comprises a surface layer from 1 to 50 microns thick comprising from 90 to 10 weight % of a spinel of the formula Mn_{.x}Cr₃₋ₓO₄ wherein x is from 0.5 to 2, from 10 to 90 weight % of oxides of Mn, Si selected from the group consisting of MnO, MnSiO₃, Mn₂SiO₄ and mixtures thereof.
